(19) United States
(12) Patent Application Publication
(10) Pub. No.: US 2007/0083153 A1
(43) Pub. Date: Apr. 12, 2007

(54) INFUSION SYSTEM FOR ADMINISTRATION OF A LIQUID MEDICATION

(76) Inventor: Hans-Peter Haar, Wiesloch (DE)

Correspondence Address: BARNES & THORNBURG LLP, 11 SOUTH MERIDAN STREET, INDIANAPOLIS, IN, 46204, US

(21) Appl. No.: 11/536,450

(22) Filed: Sep. 28, 2006

(30) Foreign Application Priority Data
Oct. 8, 2005 (DE) EP 05 021 982.3

(51) Int. Cl. A61M 31/00

(52) U.S. Cl. 604/67; 128/903

(57) ABSTRACT

An infusion system for administration of a liquid medication may comprise a pump configured to pump the liquid medication through an infusion tube into the body of a patient, and a pressure sensor configured to monitor pressure in the infusion tube. The system may further comprise a control unit configured to control the pump. The infusion tube may carry a data line for communication of the control unit with either of the pump and the pressure sensor. The pressure sensor may monitor pressure in the infusion tube by detecting any pressure-effected expansion of a section of the infusion tube. The pressure sensor may comprise a first sensor surface arranged opposite to a second sensor surface that is carried by the section of the infusion tube. The fist and second sensor surfaces together may form a capacitor defining a capacitance that changes upon expansion of the section of the infusion tube.

### CROSS-REFERENCE TO RELATED APPLICATION

This application is a U.S. counterpart application of, and claims priority to, European Application Serial No. EP05021982.3, filed Oct. 8, 2005.

### BACKGROUND AND SUMMARY

The invention relates to an infusion system for administration of a liquid medication, in particular insulin, comprising a pump that can be used to pump the medication through an infusion tube into the body of a patient, and a control unit for controlling the pump. An infusion system of this type is known, for example, from US 2005/0137573 A.

Many diabetics require external dosing of insulin to regulate their blood glucose levels. It is customary for these insulin administrations to be given several times daily in the form of injections. Although continuous insulin administration is advantageous from a medical point of view, infusion systems for continuous administration of insulin have not become established for a variety of reasons. On the one hand, the convenience of wear of known infusion systems is insufficient, on the other hand, a user must check the proper function of his infusion system at regular intervals in order to assure that the desired dose of insulin is, in fact, being administered.

Analysis of the pressure in the infusion tube can be used to determine whether or not the medication is being pumped into the body of the patient at the desired infusion rate. For example a clot at the exit opening of the infusion tube may lead to less than the set quantity of medication being delivered to the body or the delivery of medication ceasing altogether due to an obstruction at the exit opening. This leads to an increase of the pressure in the infusion tube that can be detected via a pressure sensor. Conversely, i.e. a decrease of pressure in the infusion tube may indicate that the medication is exiting from the infusion tube without entering the body of the patient, for example due to leakage or because a catheter has become undone.

If pressure data determined from the pressure sensor indicate that the pressure of the liquid deviates from a reference value by more than a preset threshold value, this can be displayed to the user by a signaling facility, for example by vibration or an acoustic signal. A user can therefore be alerted to a malfunction of the infusion system such that the malfunction can be remedied.

The control unit may generate, as a function of pressure data that were determined by the pressure sensor, control signals for controlling the pump. This is the case, since upon obstruction of the infusion tube there is a risk that a major quantity of medication accumulates if the pressure of the liquid in the infusion tube rises for an extended period of time of several hours. If the obstruction ultimately is removed by the action of the increased liquid pressure, the entire accumulated quantity of medication is delivered to the body in a short period of time. In the case of the medication being insulin, this can lead to a hazardous decrease of the blood glucose content. A malfunction of the infusion system of this type can be prevented by designing the control unit such that the pump is turned off if it is found, on the basis of pressure data determined by the pressure sensor, that the liquid pressure exceeds a threshold value. Upon less pronounced changes of the pressure, suitable control signals of the control unit can increase or decrease the pumping rate according to need.

To make carrying the device convenient it is desirable to use an external control unit. The parts required for the pump may then be minimized such that a particularly compact and light-weight pump can be implemented. The operational safety of an infusion system of this type can be increased by the infusion tube being the carrier of a data line for communication of the control unit with the pump and/or the pressure sensor. It is thereby feasible to exclude the inherent risk of wireless communication, which is that the control unit of a first patient incorrectly controls the pump of a second, nearby patient.

An infusion system for administration of a liquid medication, in particular insulin, may comprise a pump that can be used to pump the medication through an infusion tube into the body of a patient, and a control unit for controlling the pump, characterized in that the infusion tube is carrier of a data line for communication of the control unit with the pump and/or a pressure sensor that monitors the liquid pressure in the infusion tube.

The infusion tube may comprise a layer of metal. The layer of metal can serve as sensor surface of a pressure sensor operating according to the principles of capacitance that is used to detect any expansion of the infusion tube. Moreover, the layer of metal can be used as an electrical data line via which the control unit, the pump and/or the pressure sensor can exchange data.

### BRIEF DESCRIPTION OF THE DRAWINGS

FIG. 1 shows one embodiment of an infusion system for administration of a liquid medication; and

FIG. 2 shows a schematic representation of the pressure sensor of the infusion system shown in FIG. 1.

### DESCRIPTION OF ONE OR MORE ILLUSTRATIVE EMBODIMENTS

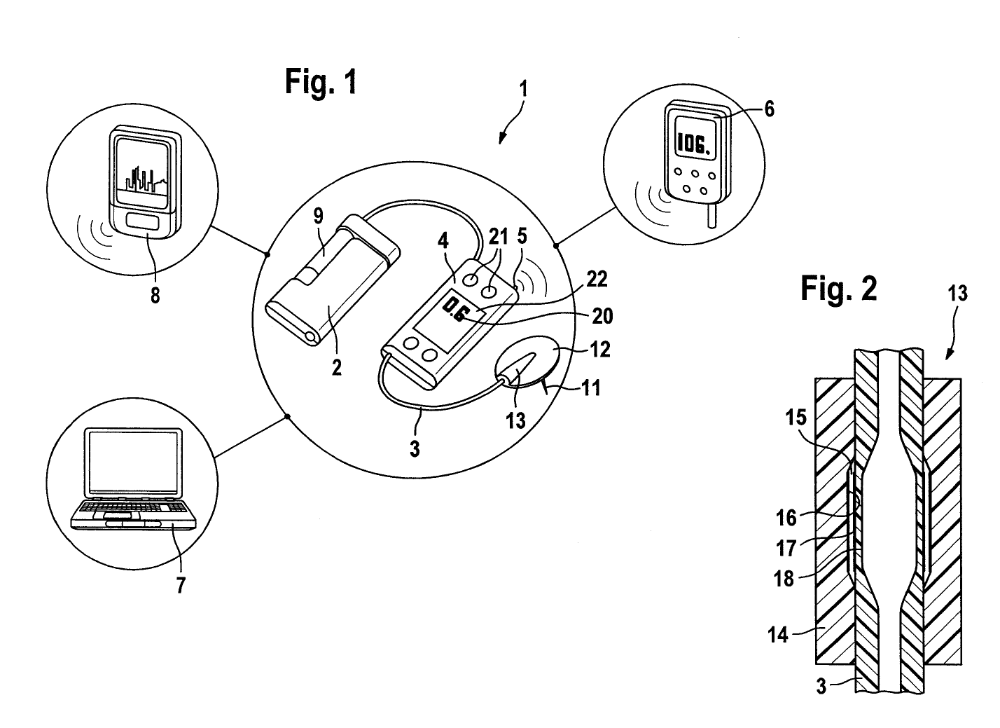

The infusion system 1 for administration of a liquid medication, in particular insulin, shown in FIG. 1, comprises a pump 2 that can be used to pump the medication through an infusion tube 3 into the body of a patient, and an external control unit 4 for controlling the pump 2. The external control unit 4 comprises an infrared interface 5 with an infrared emitter and an infrared receiver for communication with external devices. A blood glucose measuring device 6, a PC 7, and a PDA (Personal Data Assistant) 8 are shown as examples of external devices.

A medication cartridge 9 containing the medication to be administered can be placed in the pump 2. The pump 2 and the control unit 4 each have an internal power supply in the form of commercial batteries, illustratively rechargeable batteries.

The infusion tube 3 exits into a catheter 11 that projects into the body of a patient according to its purpose. The catheter 11 is fixed by common technique using an adhesive pad 12 that is attached by adhesive to the skin of the patient. In addition, the adhesive pad 12 carries a pressure sensor 13 for monitoring of the pressure in the infusion tube 3.

The structure of the pressure sensor 13 is shown schematically in FIG. 2. The pressure sensor 13 comprises a housing 14 through which the infusion tube 3 is guided. Inside the housing 14 is provided a chamber 15 whose internal wall forms a first sensor surface 16 opposite from a second sensor surface 17 that is carried by the tube section 18 of the infusion tube 3 that resides inside the chamber. In the exemplary embodiment shown, the first sensor surface 16 surrounds, in the shape of a cylinder, the second sensor surface 17 that is formed by an external surface of the tube section 18. The two sensor surfaces 16, 17 form a capacitor whose capacitance changes upon expansion of the tube section 18. The sensor surfaces 16 and 17 are layers of metal. Both the sensor housing 14 and the infusion tube 3 are otherwise made of plastic.

The pressure sensor 13 is designed such that the pressure of the liquid is being monitored by detecting any pressure-effected expansion of the tube section 18 of the infusion tube 3. An expansion of the tube section 18 leads to a change in the capacitance of the capacitor formed by the sensor surfaces 16, 17. The capacitor is part of an oscillating electrical circuit (not shown) whose resonance frequency is a function of the capacitance of the capacitor 16, 17. In order to determine the pressure, the frequency of the oscillating circuit is measured and the capacitance of the capacitor 16, 17 is determined therefrom or the associated pressure is determined directly via a suitable calibration curve.

The tube section 18 whose expansion is detected by the pressure sensor 13 has a thinner wall than neighboring tube sections. This allows the measuring sensitivity of the pressure sensor 13 to be increased. Illustratively, the infusion tube 3 may have an outer diameter of 150 to 300 μm, and in one exemplary embodiment an outer diameter of 180 to 220 μm. The infusion tube 3 may illustratively have a wall thickness of 50 to 200 μm, and in one exemplary embodiment a wall thickness of 70 to 150 μm. In another exemplary embodiment, the tube 3 may have a wall thickness of 80 to 120 μm. The wall thickness of the tube section 18 whose expansion is detected by the pressure sensor 13 is illustratively less than 60%, or alternatively only 20% to 50%, or alternatively still 30% to 40%, of the wall thickness of neighboring tube sections.

Pressure data determined by the pressure sensor 13 are transmitted to and analyzed by the control unit 4. The control unit 4 generates, as a function of pressure data that were determined by the pressure sensor 13, control signals for controlling the pump 2. In this context, the control unit 4 is illustratively designed such that the pump 2 is turned off if it is found, on the basis of pressure data determined via the pressure sensor 13, that the pressure of the liquid exceeds a threshold value.

The external control unit 4 further comprises a signaling and display facility 20 that can be used to alert a user by suitable signals to the fact that the pressure deviates from a nominal value by more than a preset threshold value. Such deviation suggests malfunction of the infusion system 1 and is displayed to the user via the signaling and display facility 20, by an acoustic signal and/or by vibration.

The external control unit 4 further comprises operating elements 21 in the form of keys that can be used in combination with a display 22 of the signaling and display facility 20 to operate the infusion system 1 and, for example, set a desired infusion rate.

In the infusion system 1 shown, the infusion tube 3 is the carrier of a data line for communication of the control unit 4 with the pump 2 and the pressure sensor 13. The data line also serves for supplying power to the pressure sensor 13. The data line is illustratively an infrared data line. For example, the infusion tube 3 can be the carrier of an optical fiber that is embedded into or arranged on the tube 3. For this purpose, the control unit 4 and the pump 2 each illustratively contain an infrared laser, a VCSEL laser (Vertical Cavity Surface Emitting Laser), for generating data pulses, and a wavelength-adjusted photodiode for receiving the data pulses.

Alternatively, the data line can be an electrical data line. Embedding an electrical conductor, illustratively a layer of metal, in the infusion tube 3 allows an electrical data line to be implemented with little effort. Illustratively, the data line is capacitively coupled to the pump 2. Capacitive coupling is advantageous in that no opening that would have to be sealed needs to be generated in the hermetically sealed housing of the pump 2 to allow the data line to be guided through. Capacitive coupling can be implemented via a metallic infusion tube 3 and an electrode surface on an internal surface of a housing of the pump 2. The electrode surface of the pump 2 and the metallic infusion tube 3 then form a capacitor via which data can be transmitted. Capacitive coupling of the data line can be utilized also for the control unit 4 and the pressure sensor 13.

Illustratively, a glucose sensor allowing for continuous measurement of the glucose concentration in the blood or interstitial fluid of the patient is integrated into the adhesive pad 12 and the catheter 11. Measuring data determined by the glucose sensor are transmitted to and analyzed by the control unit 4 via the data line formed by the infusion tube 3. The measuring values can then be displayed using the display 22. Ilustratively, the control unit 4 generates, as a function of the measuring data that was determined by the glucose sensor, control signals for controlling the pump 2 such that the insulin infusion rate can be adapted to the current need of the patient.

The operating principle of the pump 2 is illustratively based on a piston element (not shown) being moved into the cartridge 9 by a pump drive such that the liquid contained in the cartridge 9 is pressed into the infusion tube 3. Illustratively, the piston element is pliable, for example a helical spring, such that the overall length of the pump 2 can be reduced to a minimum. For protection of the pump 2 from moisture it is desirable to hermetically encapsulate the piston element using a rubber socket (not shown).

In order to increase the convenience for the user, the external control unit 4 is slidable to any position along the infusion tube. This is attained by guiding the infusion tube 3 through a channel in the control unit 4. Furthermore, the convenience for the user can be increased by the infusion system 1 containing multiple, illustratively two, pumps 2 that can be used in an alternating fashion. The pump that is not in use at a given time can then be placed in a base station in order to charge its rechargeable batteries.

1-11. (canceled)

12. An infusion system for administration of a liquid medication comprising:
a pump configured to pump the liquid medication through an infusion tube into the body of a patient, and
a pressure sensor configured to monitor pressure in the infusion tube by detecting any pressure-effected expansion of a section of the infusion tube, the pressure sensor comprising a first sensor surface arranged opposite to a second sensor surface that is carried by the section of the infusion tube, the fist and second sensor surfaces together forming a capacitor defining a capacitance that changes upon expansion of the section of the infusion tube.

13. The infusion system of claim 12 further comprising a control unit configured to control the pump.

14. The infusion system of claim 13 wherein the control unit is configured to control the pump based on the pressure in the infusion tube by generating, as a function of the capacitance of the pressure sensor, control signals that control operation of the pump.

15. The infusion system of claim 14 wherein the control unit is configured to turn off the pump if the capacitance of the pressure sensor indicates that the pressure of the liquid medication in the infusion tube exceeds a threshold value.

16. The infusion system of claim 12 wherein the section of the infusion tube has a lesser wall thickness than neighboring tube sections.

17. The infusion tube of claim 16 wherein the section of the infusion tube has a wall thickness that is less than 60% of the wall thickness of neighboring wall sections.

18. The infusion system of claim 12 wherein the liquid medication is insulin.

19. The infusion system of claim 13 wherein the control unit comprises an interface for communication with at least one other device.

20. The infusion system of claim 19 wherein the interface is a wireless interface.

21. The infusion system of claim 19 wherein the at least one other device is a blood glucose measuring device.

22. The infusion system of claim 13 wherein the infusion tube carries a data line for communication of the control unit with either of the pump and the pressure sensor.

23. The infusion system of claim 22 wherein the data line is an infrared data line.

24. The infusion system of claim 22 wherein the data line is an electrical data line, and the electrical data line is capacitively coupled to the control unit and to either of the pump and the pressure sensor.

25. The infusion system of claim 24 wherein the electrical data line comprises a layer of metal carried by the infusion tube.

26. An infusion system for administration of a liquid medication comprising:
a pump configured to pump the medication through an infusion tube into the body of a patient,
a control unit configured to control the pump, and
a pressure sensor configured to monitor pressure in the infusion tube,
wherein the infusion tube carries a data line for communication of the control unit with either of the pump and the pressure sensor.

27. The infusion system of claim 26 wherein the data line is an infrared data line.

28. The infusion system of claim 26 wherein the data line is an electrical data line, and the electrical data line is capacitively coupled to the control unit and to either of the pump and the pressure sensor.

29. The infusion system of claim 28 wherein the electrical data line comprises a layer of metal carried by the infusion tube.

30. The infusion system of claim 26 wherein the control unit comprises an interface for communication with at least one other device.

31. The infusion system of claim 30 wherein the interface is a wireless interface.

32. The infusion system of claim 30 wherein the at least one other device is a blood glucose measuring device.
